(19) United States
(12) Patent Application Publication
(10) Pub. No.: US 2017/0252168 A1
(43) Pub. Date: Sep. 7, 2017

(54) FULL CERAMIC KNEE JOINT PROSTHESIS HAVING POROUS REAR FACE FACING THE BONE

(71) Applicant: CeramTec GmbH, Plochingen (DE)

(72) Inventors: Roman PREUSS, Kirchheim unter Teck (DE); Marita RASCHKE, Leonberg (DE); Peter STRÖTGEN, Königsfeld (DE); Tobias WEISS, Ebersbach (DE)

(73) Assignee: Ceram Tec GmbH, Plochingen (DE)

(21) Appl. No.: 15/324,458

(22) PCT Filed: Jul. 1, 2015
(86) PCT No.: PCT/EP2015/064924

§ 371 Date: May 19, 2017

(30) Foreign Application Priority Data
Jul. 9, 2014 (DE) 10 2014 213 356.4

(51) Int. Cl. A61F 2/30; A61L 27/16; A61L 27/10; A61L 27/30; A61L 27/34; A61L 27/32; A61L 27/52; A61F 2/38; A61L 27/56

(52) U.S. Cl. CPC A61F 2/30771; A61F 2/3859; A61F 2/389; A61F 2/3094; A61L 27/16; A61L 27/10; A61L 27/56; A61L 27/34; A61L 27/32; A61L 27/52; A61L 27/306; A61F 2002/30013; A61F 2310/00179; A61F 2002/30968; A61F 2310/00928; A61F 2310/00976; A61F 2310/00796; A61F 2002/3093; A61L 2430/24

(57) ABSTRACT

The invention relates to a knee joint prosthesis comprising a femoral component (1) and a tibial component (2), each having a front face constituting the joint and a rear face (4) facing the bone, and a polyethylene (PE) liner (3). In order to allow the knee joint prosthesis to be implanted without metal and without cement, the femoral component (1) and the tibial component (2) consist of a full ceramic material and both components (1, 2) have integrated porous osseointegrative rear faces (4) facing the bone.

The invention relates to a knee joint prosthesis having a femoral component and a tibial component, each with a joint-forming anterior face and a bone-facing rear face, and a polyethylene (PE) liner.

The current clinical prior art for knee joint replacement provides over 90% cemented endoprostheses. The bone cement initially adheres very well to the highly roughened surfaces of the metallic endoprostheses (CoCrMo—or Ti alloys), but over time becomes detached from the component due to the effect of the watery surroundings in the human body (so-called “debonding”). Due to micro-movements of the knee endoprostheses on the bone cement plug and the progressing subcritical crack growth of the bone cement due to dynamic loading of the joint, the bone cement is broken over time. Wear particles appear, which on the one hand enter the surrounding tissue, and on the other can also migrate into the sliding surface of the joint, and there can intensively wear down the polyethylene (PE) liner. The bone cement and PE wear that occur usually lead to a biological reaction of the surrounding tissue. The consequences are local inflammation and osteolysis (disappearance of the bone), which can again lead to aseptic loosening of the implant. This mechanism is inevitable for cemented knee endoprostheses, and also explains why the average lifespan of a cemented knee joint endoprosthesis is only 10 to 20 years.

Cement-free endoprostheses constitute an alternative that has great potential for longer prosthesis lifetimes. Presently stabilized in the market are metallic knee joint endoprostheses that are frequently provided with a likewise metallic osseointegrative coating, which allows the direct ingrowth of the bone into the implant surface. Through the direct growth and ingrowth of the bone on or in the implant surface, a long-lasting stable bond is ensured between the implant and bone, and the lifetimes of the prostheses can be greatly increased in comparison with cemented variants (up to 30 years). Cementless metallic endoprostheses are exposed to greater wear than would be the case with a ceramic variant. A further great disadvantage is the use of metal per se, as currently more and more people develop intolerances to the metals that are used, and a metallic knee joint prosthesis is not suited for persons with allergies.

The use according to the invention of directly implantable full ceramic knee joint components (femoral and tibial component, combined in the entire knee system with a PE liner) with an integrated porous osseointegrative rear face facing the bone is an advantageous novelty.

Such metal-free knee joint components combine the advantages of a biocompatible, allergy-appropriate material and low wear of the sliding pair with excellent osseointegration.

EP 0 542 815 B1 and EP1 268 364 A1 are mentioned as prior art.

A knee joint prosthesis with a femoral component and a tibial component, each having a joint-forming anterior face and a rear face facing the bone and a polyethylene (PE) liner, is characterized in that the femoral component and the tibial component consist of a full ceramic material, wherein both components have a porous osseointegrative rear face facing the bone.

In an embodiment according to the invention, the femoral component and the tibial component are each manufactured from a ceramic sintered molded article, the sintered molded article having a porous layer on its rear face facing the bone.

In another embodiment according to the invention, the porous rear face facing the bone is created by application of a ceramic slurry and place-holders (pore-formers) on the main body (component) in the green state. By means of a sintering process, the layer is bonded to the main body. By burning off the place-holders during sintering, an open-pored structure is created with rugged surface that advantageously supports the accretion and ingrowth of the bone. See also EP 1 268 364 A1 in this regard.

The ceramic slurry consists of the same material as the main ceramic body. The entire implant in this way is completely free of metals including the coating, and advantageous with regard to allergic reactions.

In a further embodiment according to the invention, the porous rear face facing the bone is formed by foaming of a ceramic slurry and place-holders (pore-formers) on the main body (component) in the green state. By means of the sintering process, a bond is achieved between the layer and the main body and an open-pored structure is created with rugged surface, which advantageously supports the accretion and ingrowth of the bone. This type of coating in combination with a ceramic base article is likewise metal-free and advantageous with respect to allergic reactions.

The porous rear face facing the bone can also be created by joining of a dense molded article with a porous ceramic mold in the sintered state. The porous mold can be created by foaming of ceramic slurry and sintering or infiltration of a porous substrate material with slurry and sintering. The joining is preferably carried out by soldering or gluing. This type of coating in combination with a ceramic main body is advantageously metal-free with respect to allergic reactions.

The integrated porous osseointegrative rear face facing the bone can according to the invention also be created by 2c-injection molding (dense phase and porous or pore-forming phase) and sintering. This type of coating in combination with a ceramic main body is metal-free.

The porous rear face facing the bone can preferably acquire additional functionality, such as higher integration speed or antibacterial effects. Integration speed is understood to be the time of ingrowth with the bone. Preferably this can be achieved with bioglasses, hydroxylapatite, functionalized proteins, or hydrogels.

In a further embodiment according to the invention, the porous rear face facing the bone can be created by application of a porous metallic biocompatible layer with the already sintered ceramic main body (see EP 1 052 949 B1).

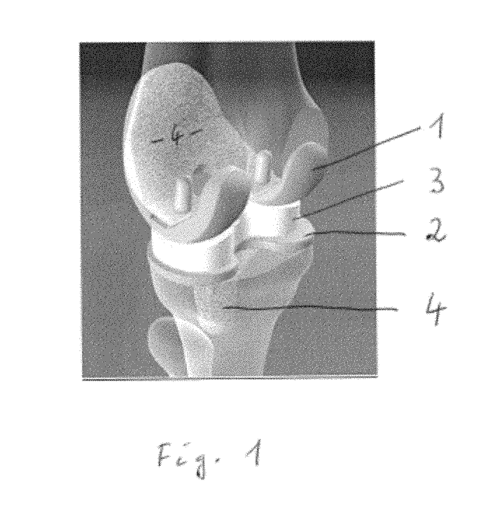

FIG. 1 shows a total ceramic knee joint endoprosthesis consisting of a femoral component 1, a tibial component 2, and intermediate polyethylene (PE) liner 3 in implanted state. The porous rear face facing the bone is marked by the reference sign 4.

1. A knee joint prosthesis with a femoral component and a tibial component, each with an anterior face forming the joint and a rear face facing the bone and with a polyethylene (PE) liner, wherein the femoral component and the tibial component consist of a full ceramic and both components have an integrated porous osseointegrative rear face facing the bone.

2. The knee joint prosthesis according to claim 1, wherein the femoral component and the tibial component are each manufactured from a ceramic sintered molded article.

3. The knee joint prosthesis according to claim 2, wherein the sintered molded articles have a porous layer on their rear face.

4. The knee joint prosthesis according to claim 1, wherein the porous rear face is formed by application of a ceramic slurry and place-holders (pore-formers) in the green state and burning off of the place-holders during sintering.

5. The knee joint prosthesis according to claim 1, wherein the porous layer is created by foaming of a ceramic slurry in the green state and subsequent sintering.

6. The knee joint prosthesis according to claim 1, the porous rear face is created by joining of a dense molded article with a porous ceramic mold in the sintered state.

7. The knee joint prosthesis according to claim 6, wherein the porous mold is created by foaming of ceramic slurry and sintering or infiltration of a porous substrate material with slurry and sintering.

8. The knee joint prosthesis according to claim 6, wherein the joining is carried out by soldering or gluing.

9. The knee joint prosthesis according to claim 1, wherein the integrated porous osseointegrative rear face is created by 2c-injection molding (dense phase and porous or pore-forming phase) and sintering.

10. The knee joint prosthesis according to claim 1, wherein the porous rear face acquires an additional functionality, such as higher integration speed or antibacterial effects, through further coating.

11. The knee joint prosthesis according to claim 10, wherein the porous rear face is coated with bioglasses, hydroxylapatite, functionalized proteins, or hydrogels.
